(19) United States
(12) Patent Application Publication
(10) Pub. No.: US 2021/0275344 A1
(43) Pub. Date: Sep. 9, 2021

(54) Urination Collection Nozzle and System for Urine Discarding

(71) Applicant: Douglas Wing, St. Clair Shores, MI (US)

(72) Inventor: Douglas Wing, St. Clair Shores, MI (US)

(21) Appl. No.: 17/192,620

(22) Filed: Mar. 4, 2021

Related U.S. Application Data
(60) Provisional application No. 62/985,038, filed on Mar. 4, 2020.

(51) Int. Cl. A61F 5/451; A61F 5/44

(52) U.S. Cl. CPC A61F 5/451; A61F 5/4405; A61F 5/4401

(57) ABSTRACT

A urine collection system is provided. The system includes a nozzle having a first end that secures to a hose and a second end that is sized and shaped to receive urine from genitalia therethrough. The nozzle width tapers inwardly from the second end to the first end, forming a funnel configuration. The nozzle further includes a plurality of apertures that are sized to retain a comfortable vacuum seal in the funnel. The hose is operably connected to a vacuum source, such as a wet-dry vacuum, which evacuates urine from the nozzle and hose. At least one bladder leakage pad is housed within the vacuum source that absorbs the urine. In other embodiments, the hose is secured directly to a plumbing system, and includes a vacuum source and a check valve. The device provides a sanitary means for an individual to urinate.

### CROSS REFERENCE TO RELATED APPLICATIONS

This application claims the benefit of U.S. Provisional Application No. 62/985,038 filed on Mar. 4, 2020. The above identified patent application is herein incorporated by reference in its entirety to provide continuity of disclosure.

### BACKGROUND OF THE INVENTION

The present invention relates to a urine collection device. More specifically, the present invention provides a urine collection nozzle that uses the suction from a vacuum source to collect the urine from an individual while they are laying horizontally or in a standing position.

As people get older, their bladder can go through changes. The elastic tissue that comprises the bladder may harden over time, making the tissue less malleable. When the bladder tissue becomes less elastic, it cannot store the same quantity of urine as when it had a high elasticity. When the individual cannot store a lot of urine in their bladder, it requires them to have to empty their bladder more frequently. Even when the individual is sleeping, they can have a high frequency of inconsistent bathroom use.

Bathroom use during the night can be a difficult inconvenience for many individuals. With many of the people that must use the bathroom during the night being of older age, they may have some physical limitation that makes it difficult for them even getting up to use the bathroom in the middle of the night. In addition to the possible physical limitation, individuals can also struggle with getting out of the bed in a dark bedroom and making their way to the bathroom. When they have to get out of the bed in a dark environment, that can lead to the individual potentially slipping or falling. If an individual that is of an older age were to slip or fall, they may suffer an injury. However, these impediments cannot deter the individual from properly emptying their bladder during the night. Failure to properly empty a bladder during the night may lead to accidents occurring within the bed. If such an accident does occur, it will force the individual to sleep in the urine which can be both unsanitary and uncomfortable.

In some cases, individuals will need the assistance of another person to help them reach the bathroom in the middle of the night. If fortunate enough to have another person living with them or to be under the care of a caretaker, some individuals might utilize such assistance in transporting to the bathroom. For the individual, waiting on the assistance of another in the middle of the night can be frustrating, embarrassing, and inconvenient to the other person. However, some individuals might be bed ridden and unable to be taken to the bathroom with just the assistance of another individual. For example, bed ridden individuals might require the aid of a wheelchair or a walker to be transported to the bathroom. In such instances, it can be cumbersome to get in and out of a wheelchair throughout the night to go to the bathroom. With such difficulty in transporting to the bathroom, alternative means that may eliminate the need to go to the bathroom to empty their bladder will be more convenient for the individual.

To avoid the suffering of an injury, people who cannot reach the bathroom will need to use an alternative means to discard their urine. When a bed ridden individual lacks the assistance of another person to help them transport to the bathroom, a popular means to discard their urine is a bed pan. A bed pan allows the individual to have a device that can be placed closed to the location of where they are sleeping. Users can urinate into the bed pan and avoid having to walk to the closest bathroom in the middle of the night. However, bed pans can be inconvenient and messy. Users normally must position their body appropriately to excrete their urine into the bed pan to avoid any urine from missing the bed pan. This can require the individual to maneuver their body in a position to both support the bed pan while urine is being placed within it and to comfortably relieve themselves of the urine. Additionally, when urine is in the bed pan it can spill on the bed or surrounding floor. Such a mess can be odorous and even can result in a slip from the individual or another person if they happen to step in the urine on the surrounding floor.

In situations where there is a staff of caretakers that are responsible for multiple individuals, it can be difficult for the staff of caretakers to timely assist all of the individuals. For example, nursing homes can be the residence for more than dozens of elderly people. Among that group of elderly people residing in a nursing home there can be a high number of them that might have smaller bladders and need help to use the bathroom at night. If the nursing home does not use bed pans because of the messes that they can make, having to serve each resident that requires the use of the bathroom will be difficult to accommodate each one at the exact time they need to use the bathroom. For facilities, such as nursing homes, that have a high number of individuals that might require frequent nighttime bathroom use, providing a reliable and simple means to allow the individuals to use the bathroom can be difficult.

Therefore, there is a defined need in the known art for a urine collection nozzle that will allow users to relieve themselves in the middle of the night without having to transport to a bathroom, without having to deal with placing their urine into a bed pan or having to rely on the assistance of another individual.

### SUMMARY OF THE INVENTION

In view of the foregoing disadvantages inherent in the known types of urine collection systems now present in the known art, the present invention provides a new urine collection system wherein urine is drawn into a nozzle which is operably connected to a vacuum source, via a hose, and is transported into the vacuum source or a plumbing system.

It is therefore an object of the present invention to provide a urine collection nozzle that collects a user's urine. The urine collection nozzle comprises a wide inlet opening that funnels to a narrow outlet opening. The wide inlet opening is designed to accept urine from the genitalia of both men and women. When the urine is drawn into the urine collection nozzle it will funnel out through the narrow outlet opening. A plurality of apertures is disposed through the urine collection nozzle. The plurality of apertures allows the users to generate additional suction force when the urine collection nozzle is coupled with a vacuum source. The urine collection nozzle is slightly angled towards the narrow outlet opening to allow for users to insert their urine while they are in a horizontal position, in addition to a vertical position.

Another aspect of the present invention is to integrate the urine collection nozzle with a vacuum source. The vacuum source will generate a suction force to draw the urine into the wide inlet opening of the urine collection nozzle and draw the urine out of the urine collection nozzle through the narrow outlet opening. When the urine is drawn out of the urine collection nozzle by the suction force, the suction force will transport the urine for discarding.

Yet another aspect of the present invention is how the urine collection systems disposes of the urine that is collected. In one embodiment of the urine collection system, the urine will be collected in the vacuum source. An internal storage compartment of the vacuum source that stores the collected material will contain at least one bladder leakage pad. The at least one bladder leakage pad will collect the urine that enters the vacuum source. Once all of the urine is collected into the vacuum source, the at least one bladder leakage pad can be discarded and a new bladder leakage pads inserted into the internal storage compartment of the vacuum source.

In another embodiment of the urine collection system, the urine will enter an internal plumbing system. A check valve will connect the urine collection nozzle to the vacuum source. The check valve will also connect the urine collection nozzle to a vent pipe that leads to the internal plumbing system. As the vacuum source draws the urine from the urine collection device, the check valve will direct the urine towards the vent pipe. In this embodiment, the urine will be led through the internal plumbing system and discarded as if the urine was placed into a toilet. Additionally, in this embodiment, there is no need to dispose of any bladder leakage pad or any other type of subsequent discarding.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1a shows a perspective view of an embodiment of the urine collection nozzle.

FIG. 1b shows a perspective view of an alternative embodiment of the urine collection nozzle.

FIG. 2 shows a perspective view of an embodiment of the urine collection system.

FIG. 3 shows a perspective view of an embodiment of the urine collection system in use.

FIG. 4 shows a perspective view of an alternative embodiment of the urine collection system.

### DETAILED DESCRIPTION OF THE INVENTION

Reference is made herein to the attached drawings. Like reference numerals are used throughout the drawings to depict like or similar elements of the urine collection nozzle and urine collection systems. The figures are intended for representative purposes only and should not be limiting in any respect.

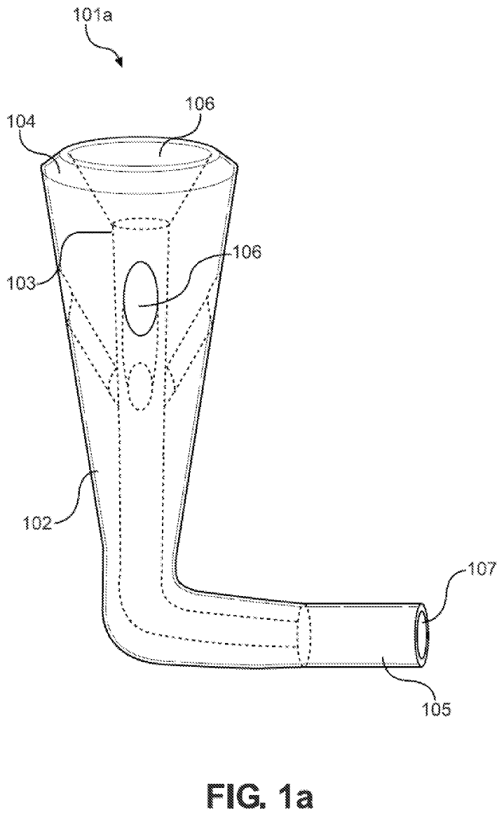

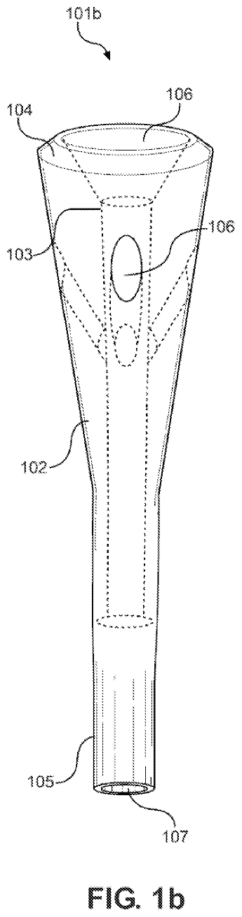

FIG. 1a and FIG. 1b show a perspective view of alternative embodiments of a urine collection nozzle. The urine collection nozzle 101a, 101b includes a conical body. The conical body further comprises an exterior face 102, an interior face 103, a working end 104, and an attaching end 105. The conical body of the urine collection nozzle 101a, 101b funnels from the working end 104 to the attaching end 105. In one embodiment, the conical body of the urine collection nozzle 101a bends at a 90-degree angle towards the attaching end 105. In another embodiment, the conical body of the urine collection nozzle 101b is vertical. The working end 104 has a wide inlet opening 106. The wide inlet opening 106 is connected to the interior face 103. The attaching end 105 has a narrow outlet opening 107. The narrow outlet opening 107 is connected to the interior face 103.

The urine collection nozzle 101a, 101b also comprises a plurality of aperture vents 108. The plurality of aperture vents 108 is disposed on the exterior face 102 of the urine collection nozzle 101a, 101b conical body. The plurality of aperture vents 108 extend through the urine collection nozzle 101a, 101b conical body until it reaches the interior face 103. The plurality of aperture vents 108 comprises an exterior opening of aperture vent 108 nearer to working end 104 than interior opening of aperture vent 108. The plurality of aperture vents 108 is downwardly angled within the conical body so that when urine is placed within the wide inlet opening 106 disposed on the working end 104, the urine will not seep out of the plurality of aperture vents 108.

When connected to the urination collection system, the attaching end 105 of the urine collection nozzle 101a, 101b will be attached to a vacuum source that creates a suction force through interior face 103 of the urine collection nozzle 101a, 101b conical body. The suction force will collect all the urine that is placed upon the wide inlet opening 106 disposed on the working end 104. The suction force will generate a vacuum seal at the wide inlet opening 106. When a user covers the plurality of aperture vents 108 disposed on the external face of the urine collection nozzle 101a, 101b, the suction force at wide inlet opening 106 disposed on the working end 104 will increase.

The wide inlet opening 106 is designed to accept urine from the genitalia of both men and women. The wide inlet opening 106 disposed on the working end 104 will accept the urine from a user's genitalia. The urine will travel along the interior face 103 of the urine collection nozzle 101a, 101b. The urine will exit the urine collection nozzle 101a, 101b and enter a urine collection system by way of the narrow outlet opening 107 disposed on the attaching end 105.

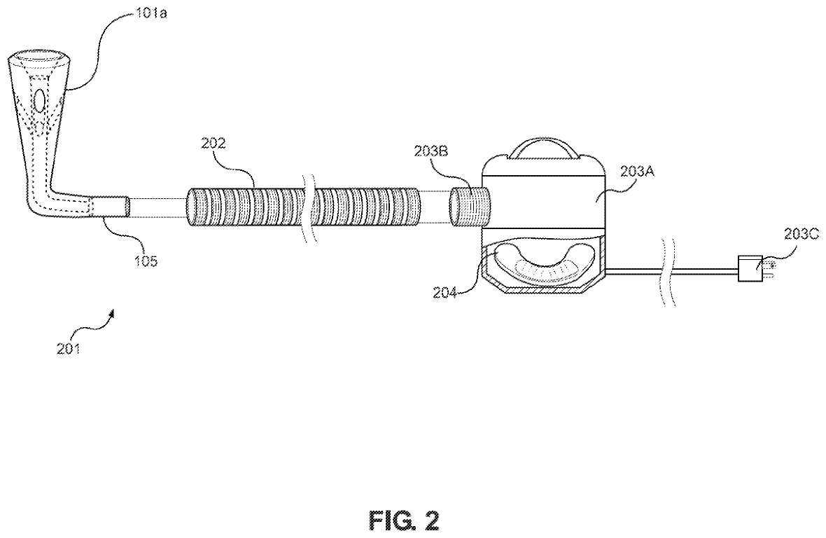

FIG. 2 shows a perspective view of an embodiment of a urine collection system. The urine collection system 201 allows a user to properly transport their urine when they are unable to get to a bathroom. The urine collection system 201 comprises a urine collection nozzle 101a, at least one hose 202, and a vacuum source 203A. The vacuum source 203A further comprises an internal storage, a power connector 203C, and an attachment inlet 203B. The attachment inlet 203B is designed to accept at least one hose 202. Each hose 202 has two opposing ends. A first end of the at least one hose 202 is attached to the attaching end 105 of the urine collection nozzle 101a. A second end of the at least one hose 202 is attached to the attachment inlet 203B disposed on the vacuum source 203A. The at least one hose 202, further comprises a body that is flexible and can be contorted in such a way that the at least one hose 202 remains attached to both the urine collection nozzle 101a and the attachment inlet 203B disposed on the vacuum source 203A. The user can manipulate the urine collection nozzle 101a to properly position it to accept the urine from their genitalia. The at least one hose 202 will remain attached to both ends when the user manipulates the position of the urine collection nozzle 101a to complete the urine collection system 201.

Included in this embodiment of the urine collection system 201, is at least one bladder leakage pad 204. The at least one bladder leakage pad 204 is placed within the internal storage of the vacuum source 203A. The at least one bladder leakage pad 204 is large enough to cover the lower surface of the internal storage of the vacuum source 203A. The internal storage of the vacuum source 203A is operably connected to the attachment inlet 203B. When an object or a liquid that enters the vacuum source 203A by way of the attachment inlet 203B, the object or liquid will be housed in the internal storage of the vacuum source 203A. In this embodiment of the urine collection system 201, the at least one bladder leakage pad 204 will absorb any liquid that enters the vacuum source 203A by way of the attachment inlet 203B. The vacuum source 203A will receive power by coupling the power connector 203C to a power source. In one embodiment of the urine collection system 201, the power source is a wall power outlet. In another embodiment of the urine collection system 201, the power source is a 110-volt outlet inside an automobile. When the vacuum source 203A receives power, it will generate the suction force through the hose 202 and to the urine collection nozzle 101a. The suction force will go through the urine collection nozzle 101a to the wide inlet opening 106. The suction force will take-in the urine from the user's genitalia. The suction force at the wide inlet opening 106 can be altered by the user. By covering the plurality of aperture vents 108 disposed on the external face of the urine collection nozzle 101, a seal is created that will increase the suction force at the wide inlet opening 106.

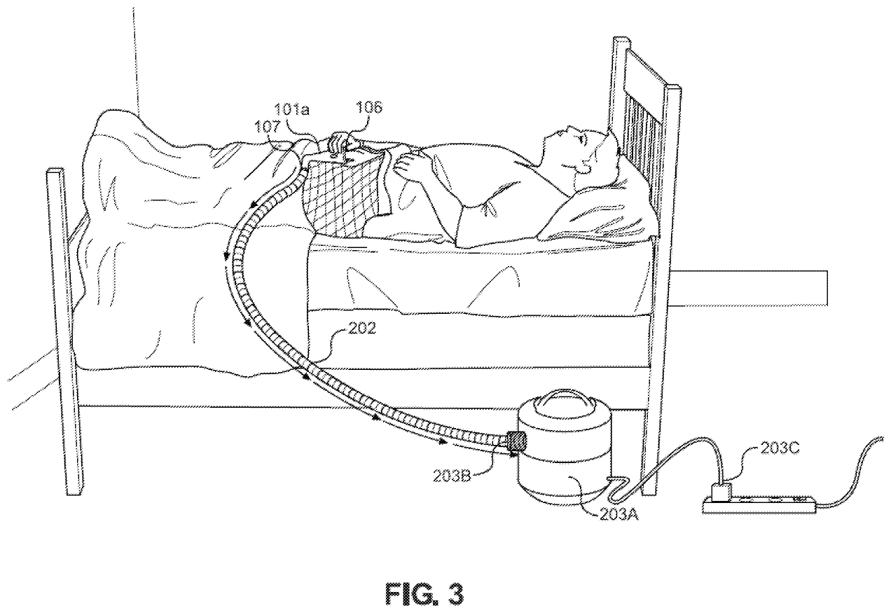

FIG. 3 shows a perspective view of an embodiment of a urine collection system in use. In this embodiment of the urine collection system 201, when the vacuum source 203A is being supplied power by the power connector 203C, the user will selectively turn on the suction force that will occur at the wide inlet opening 106 of the urine collection nozzle 101a. The user can then place their urine into the wide inlet opening 106 of the urine collection nozzle 101a. When the urine leaves the urine collection nozzle 101a by the narrow outlet opening 107, the urine will be transported to the attachment inlet 203B of the vacuum source 203A by the at least one hose 202. When the urine enters the vacuum source 203A by way of the attachment inlet 203B, said urine will then be deposited into the internal storage of the vacuum source 203A. The urine will be absorbed by the at least one bladder leakage pad 204 that is placed within the internal storage. When all the urine has been deposited in the internal storage of the vacuum source 203A the user can disconnect the power connector 203C from a power source and remove the at least one bladder leakage pad that have absorbed the urine and replace them with a new at least one bladder leakage pad. When the new at least one bladder leakage pad 204 is placed within the internal storage of the vacuum source 203A, the urine collection system 201 will be ready for the subsequent use from the user.

This embodiment of the urine collection system 201, can have alternative uses. Other uses of this embodiment of the urine collection system 201 include removing dirt and debris from floors, carpets, rugs, and upholstery. Additionally, this embodiment of the urine collection system 201 may be used to clean up urine, or any liquid, that is formed on the floor from a domesticated animal or from a spill.

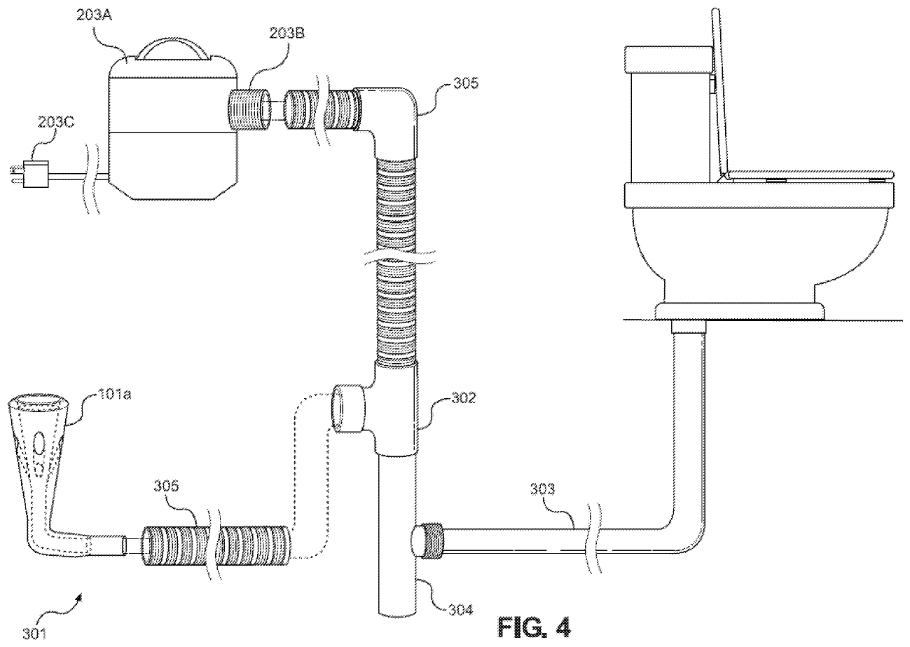

FIG. 4 shows a perspective view of an alternative embodiment of a urine collection system. The urine collection system 301 allows a user to properly transport their urine when they are unable to get to a bathroom. The urine collection system 301 comprises a urine collection nozzle 101a, a plurality of hoses 305, a vacuum source 203A, and a one-way check valve 302. The vacuum source 203A further comprises a power connector 203C and an attachment inlet 203B. The attachment inlet 203B is designed to accept at least one hose 305. Each hose 305 has two opposing ends. A first end of a first hose 305 is attached to the attaching end 105 of the urine collection nozzle 101a. A first end of a second hose 305 is attached to the attachment inlet 203B disposed on the vacuum source 203A. A second end of the first hose 305 is coupled with a second end of the second hose 305. Additionally, the one-way check valve 302 is operably connected below the coupling of the first hose 305 and the second hose 305. The one-way check valve 302 is also connected to a vent pipe 303. The vent pipe 303 is further connected to a sewage piping system 304.

Included in this embodiment of the urine collection system 301 is the one-way check valve 302. The one-way check valve 302 is coupled to a vent pipe 303. The vent pipe 303 connects any toilet or plumbing appliance to a sewage piping system 304. The configuration is such that the first hose 305 attached to the urine collection nozzle 101a is above the one-way check valve 302. Moreover, the vacuum source 203A is oriented above the urine collection nozzle 101a. In this configuration of the urine collection system 301, when the vacuum source 203A is being supplied power by the power connector 203C, the vacuum source 203A will draw air from the urine collection nozzle 101a towards the vacuum source 203A creating a suction force at the wide inlet opening 106 of the urine collection nozzle 101a. Due to the orientation of the one-way check valve 302, the suction force will not draw air from the vent pipe 303, a plumbing appliance, or the sewage piping system 304. When a user inserts their urine into the urine collection nozzle 101a, the urine will be transported through the first hose 305 towards the one-way check valve 302. Due to the orientation of the vacuum source 203A with respect to the urine collection nozzle 101a, gravity will prevent the urine from traveling through the second hose 305 towards the vacuum source 203A. When power is no longer supplied to the vacuum source 203A, the urine will pass through the one-way check valve 302 and enter the vent pipe 303. When the urine enters the vent pipe 303, it will be transported to the sewage piping system 304. In this embodiment of the urine collection system 301, no individual is required to perform any additional steps to discard the urine from the urine collection system 301.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A urine collection nozzle, comprising:
a conical body;
the conical body includes an exterior face, an interior face, a working end, and an attaching end;
a plurality of apertures disposed on the exterior face of the conical body; and
wherein the plurality of apertures is connected to a corresponding aperture disposed on the interior face of the conical body by a channel.

2. The urine collection nozzle of claim 1, wherein the conical body is hollow with a channel from the working end to the attaching end.

3. The urine collection nozzle of claim 1, wherein the conical body can be curved below the plurality of apertures where in the attaching end is perpendicular to the working end.

4. The urine collection nozzle of claim 1, wherein the conical body comprises a wide inlet opening and a narrow outlet opening.

5. The conical body of claim 3, wherein the wide inlet opening is disposed on the working end and the narrow outlet opening is disposed on the attaching end.

6. A urine collection system, comprising:
the urine collection nozzle;
at least one hose, wherein each hose comprises a body and a pair of opposing ends;
a vacuum source;
wherein the vacuum source comprises an attachment inlet and an internal storage compartment;
wherein one end of a first hose is connected to the urine collection nozzle and the other end of the first hose is connected to the attachment inlet disposed on the vacuum source; and
at least one bladder leakage pad.

7. The urine collection system of claim 5, wherein the at least one bladder leakage pad is housed in the internal storage compartment of the vacuum source.

8. The urine collection system of claim 5, wherein the vacuum source further comprises a power connector.

9. The urine collection system of claim 5, wherein the body of the at least one hose is flexible.

10. A urine collection system, comprising:
the urine collection nozzle;
a plurality of hoses, wherein each hose comprises a body and a pair of opposing ends;
wherein the urine collection nozzle is coupled with a first hose;
a vacuum source;
wherein the vacuum source is coupled with a second hose;
wherein the first hose and the second hose are coupled together to form a coupling.
a one-way check valve; and
a one-way check valve;
wherein the coupling is operably connected to the one-way check valve;
a vent pipe, wherein the vent pipe is connected to a sewage piping system; and
wherein the one-way check valve is operably connected the vent pipe.

11. The urine collection system of claim 10, wherein the vacuum source further comprises an attachment inlet and an internal storage compartment.

12. The urine collection system of claim 10, wherein the body of the plurality of hoses is flexible.

13. The urine collection system of claim 10, wherein a first end of the first hose is affixed to the urine collection nozzle and a first end of the second hose is affixed to the vacuum source.

14. The urine collection system of claim 10, wherein the coupling is operably connected to a second end of the first hose and a second end of the second hose.

15. The urine collection system of claim 10, wherein the vacuum source is configured above the urine collection nozzle.

16. The urine collection system of claim 10, wherein the one-way check valve is configured below the coupling.

17. The urine collection system of claim 10, wherein the one-way check valve is configured above the vent pipe.
